(19) United States
(12) Patent Application Publication
(10) Pub. No.: US 2024/0279294 A1
(43) Pub. Date: Aug. 22, 2024

(54) p53 VARIANT WITH IMPROVED LIQUID-LIQUID PHASE SEPARATION ABILITY AND ACTIVITY AND USE THEREOF

(71) Applicant: Zhejiang University, Hangzhou City (CN)

(72) Inventors: Guozhen WANG, Hangzhou City (CN); Yan LIU, Hangzhou City (CN)

(21) Appl. No.: 18/582,271

(22) Filed: Feb. 20, 2024

(30) Foreign Application Priority Data
Feb. 20, 2023 (CN) 202310138578.2

(51) Int. Cl. C07K 14/47; A61K 31/4709; A61K 31/519; A61K 38/00; A61P 35/00

(52) U.S. Cl. CPC C07K 14/4746; A61K 31/4709; A61K 31/519; A61P 35/00; A61K 38/00

(57) ABSTRACT

This disclosure provides a p53 variant with improved liquid-liquid phase separation ability and activity and use thereof. Compared with wild-type p53, the novel p53 variant has following advantages: 1) stronger liquid-liquid phase separation ability; 2) stronger transcriptional activation ability for p53 target genes such as CDKN1A, in tumor cells; 3) more remarkable growth inhibition effect on various tumor cells. This disclosure demonstrates that improving the liquid-liquid phase separation ability can enhance transcriptional activation activity of p53 and induce growth inhibition of tumor cells, which is of great significance for improving p53-based gene/protein therapy. Gene and protein of the p53 variant disclosed by this invention are promising anti-cancer agents with potential for clinical use. Additionally, when combined with the FGFR inhibitor TAS-120 or the Wnt pathway inhibitor IWR-1, the p53 variant shows enhanced tumor-suppressive efficacy.

### CROSS REFERENCE TO THE RELATED APPLICATIONS

This Non-provisional application claims priority under 35 U.S.C. § 119(a) on Chinese Patent Application No(s). CN 202310138578.2 filed on Feb. 20, 2023, the entire contents of which are hereby incorporated by reference.

### REFERENCE TO AN ELECTRONIC SEQUENCE LISTING

The content of the electronic sequence listing (2024-02-20-Sequence-Listing.xml; Size: 17,914 bytes; and Date of Creation: Feb. 20, 2024) is herein incorporated by reference in its entirety.

### TECHNICAL FIELD

The disclosure relates to the field of biomedicine, in particular to a p53 variant with improved liquid-liquid phase separation ability and its application in killing tumor cells.

### BACKGROUND ART

The transcription factor p53 is one of the most important tumor suppressors in cells. In over 50% of cancer, it undergoes mutations leading to a loss of its activity. Additionally, in other cancers, either the activity of p53 itself or its downstream signaling pathways are inhibited. p53, as the “guardian of the genome”, responds to stress signal to promote DNA damage repair, via activating its downstream target genes CDKN1A, PUMA, and BAX, to induce cell cycle arrest or apoptosis, thus inhibiting proliferation of tumor cells. Therefore, restoring or enhancing the activity of p53 in cancer cells is a current strategy to inhibit cancer cell proliferation.

Methods for restoring/improving the activity of p53 in cancer cells include: 1) inhibiting wild-type p53 degradation; 2) inhibiting activity of mutant p53 protein; 3) gene therapy by transforming p53 cDNA. Only therapy that has been clinically approved is the gene therapy. The gene therapy (Gendicine) by using recombinant defective adenovirus to deliver wild-type p53 for treatment of head and neck cancer has been clinically approved in China. Other gene therapies to deliver wild-type p53, such as ONYX-015, are still in clinical trials. A method of p53 gene therapy combined with an immune checkpoint inhibitor (PD-1/PD-L1 antibody) in treatment of solid tumors has entered phase II clinical trials. While gene therapy delivering wild-type p53 has been shown to kill cancer cells, the instability of wild-type p53 and gain-of-function induced by mutant p53 in cancer cells impair its efficacy, leading to limited therapeutic outcomes. Therefore, it is urgent to develop p53 variants with stronger tumor inhibition effect to improve effectiveness of p53 gene therapy.

### SUMMARY

In view of above existing problems, it is an object of the present disclosure to provide p53 variants with improved liquid-liquid phase separation ability and activity and use thereof.

The p53 variants with improved liquid-liquid phase separation ability is achieved by adding an amino acid sequence rich in positive charges and histidine to one terminus of an p53 sequence. The p53 variants have stronger liquid-liquid phase separation ability and stronger transactivation activity in cells and in vitro than the p53. A general formula of the amino acid sequence rich in positive charges and histidine is Hx(G/S/P/A/T)y(R/K)z, where H represents histidine; (G/S/P/A/T) represents a sequence composed of one or more of glycine, threonine, proline, serine and threonine; and (R/K) represents arginine or lysine, where x, y and z represent numbers of respective amino acids; x and z range from 0 to 20; y ranges from 0 to 100, preferably from 0 to 20; and x+z>5.

A linker sequence is further connected between the amino acid sequence rich in positive charges and histidine and one terminus of a p53 sequence.

The p53 has transactivation activity, DNA sequence of p53 can be but not limited to SEQ ID NO.1 or SEQ ID NO.2.

The p53 has transactivation activity for human or non-human species.

The DNA sequence of the p53 variants is shown in SEQ ID NO.3, SEQ ID NO.4, SEQ ID NO.5, SEQ ID NO.6, SEQ ID NO.7 or SEQ ID NO.8. The p53 variants have higher transactivation activity than p53.

The p53 variants can activate transcription of CDKN1A and improve mRNA levels of one or more of downstream target genes CDKN1A, MDM2, PUMA, NOXA and RRM2B.

Using of the p53 variants in preparing a drug for treating tumors, with applicable tumor types involving non-small cell lung cancer, breast cancer, neuroblastoma, osteosarcoma and human brain tumor. The drug includes a p53 variant in either gene or protein form.

A recombinant protein expression system of the p53 variants includes Escherichia coli. expression host, eukaryote cells and yeast protein expression systems. Prokaryotic expression vectors include pET24a and pET28a (+). Eukaryotic expression vectors include: 1) eukaryote cell expression vectors such as pEGFP, pEYFP, pmcherry, pRFP, pECFP, pLenti, pLX, pCMV6, pCMV3, pcDNA3 and pcDNA6B; 2) insect cell expression vectors including pAc5.1-EGFP, etc.; and 3) yeast expression vectors including pPIC3 and pPIC9.

A protein form of the p53 variants includes either a full-length form or mutant form that retains functions.

A target gene for which the p53 variants mediates tumor-cell apoptosis is FGFR3, and the p53 variant can be configured for treating tumors with high FGFR3 expression.

A drug for treating tumors is drug combined with the p53 variants, a FGFR inhibitor TAS-120 or a Wnt signal pathway inhibitor IWR-1, or a prepared composite drug, for improving its ability to kill the tumor cells at a low protein concentration.

The drug can be in a form of injection, tablet, capsule, oral liquid dosage, granule or ointment.

The disclosure has following technical effects,
(1) LLPSE-p53 (the p53 variant) innovatively designed in this application has improved liquid-liquid phase separation effect in cells and in vitro.
(2) It is specified in this application that the p53 variants with improved liquid-liquid phase separation capacity enhances transcriptional activation of p53 target gene (DKNIA and the like.
(3) The application demonstrates that LLPSE-p53 variants significantly suppress the growth of a wide range of tumor cells more effectively than p53, highlighting their considerable potential for clinical use. Additionally, it innovatively reveals for the first time that increasing the liquid-liquid phase separation propensity of p53 enhances its tumor suppression capabilities.
(4) The application demonstrates that combining low concentrations of LLPSE-p53 protein with small-molecule inhibitors targeting the Wnt signaling pathway and FGFR significantly boosts its anti-tumor effectiveness. This strategy presents an innovative and viable foundation for its clinical use in cancer treatment.

### BRIEF DESCRIPTION OF THE DRAWINGS

FIG. 1 shows comparative microscope images indicating phase separation ability of WT-p53 and a recombinant p53 protein tagged with an EGFP (Enhanced Green Fluorescent Protein) at a C-terminus (EGFP-p53) in vitro;

FIG. 2 shows test results of CDKN1A transcription after plasmids of WT-p53 or p53 with an YFP tag added at a C-terminus (YFP-p53) was transfected into a human non-small cell lung cancer cell line H1299;

FIG. 3 shows morphological confocal images of WT-p53 and LLPSE-p53 in a human renal epithelial cell line HEK293T;

FIG. 4 shows morphological confocal images of WT-p53 and LLPSE-p53 in a human non-small cell lung cancer cell line H1299;

FIG. 5 shows morphological confocal images of WT-p53(NMR) and LLPSE-p53(NMR) in a human non-small cell lung cancer cell line H1299;

FIG. 6 shows qRT-PCR test results of WT-p53 and LLPSE-p53 regulating mRNA levels of p53 downstream target genes in a human renal epithelial cell line HEK293T;

FIG. 7 shows qRT-PCR detection results of WT-p53 and LLPSE-p53 regulating mRNA levels of p53 downstream target genes in a human non-small cell lung cancer cell line H1299;

FIG. 8 shows comparative results of CDKN1A transcriptional activation in the human non-small cell lung cancer cell line H1299, following transfection with either WT-p53 or LLPSE-p53 plasmids;

FIG. 9 is a graph showing results of cell viability tests by an ATP assay system after plasmid of WT-p53 and LLPSE-p53 were transfected into a human non-small cell lung cancer cell line H1299;

FIG. 10 is a graph showing results of cell viability tests by the ATP assay system after plasmids of WT-p53 and LLPSE-p53 were transfected into a human neuroblastoma cell line SH-SY5Y;

FIG. 11 is a graph showing results of cell viability tests by the ATP assay system after plasmids of WT-p53 and LLPSE-p53 were transfected into a human osteosarcoma cell line U-2OS;

FIG. 12 is a graph showing results of cell viability tests by a CCK8 kit after plasmids of an empty vector, WT-p53 and LLPSE-p53 were transfected into a human embryonic kidney cell line HEK293T;

FIG. 13 shows microscope images of phase separation of WT-p53 and LLPSE-p53 recombinant proteins in vitro;

FIG. 14 is a graph showing results of cell viability tests by the ATP assay system after WT-p53 and LLPSE-p53 recombinant proteins were transduced into a human non-small cell lung cancer cell line H1299 for 72 h;

FIG. 15 is a graph showing results of cell viability tests by the ATP assay system after WT-p53 and LLPSE-p53 recombinant proteins were transduced into a human neuroblastoma cell line SH-SY5Y for 72 h;

FIG. 16 is a graph showing results of cell viability tests by the ATP assay system after WT-p53 and LLPSE-p53 recombinant proteins were transduced into a human breast cancer cell line SKBR3 for 72 h;

FIG. 17 is a graph showing results of cell viability tests by the CCK8 kit after WT-p53 and LLPSE-p53 recombinant proteins were transduced into a human osteosarcoma cell line U-2OS for 72 h;

FIG. 18 is a graph showing results of cell viability tests by the CCK8 kit after WT-p53 and LLPSE-p53 recombinant proteins were transduced into a human osteosarcoma cell line U-2OS for 72 h, with a control group with no recombinant protein added;

FIG. 19 is a graph showing results of cell viability tests by the CCK8 kit after WT-p53 and LLPSE-p53 recombinant proteins were transduced into a human brain tumor cell line SF126 for 72 h;

FIG. 20 is a graph showing results of cell viability tests by the CCK8 kit after WT-p53 and LLPSE-p53 recombinant proteins were transduced into a human malignant glioma cell line U-87 for 72 h;

FIG. 21 is a graph showing results of cell viability tests by the ATP assay system after a control group (Vector) and FGFR3 were knocked down respectively and then WT-p53 and LLPSE-p53 were over-expressed for 72 h;

FIG. 22 is a graph showing that the efficacy of LLPSE-p53 (SEQ ID No.3) in killing human non-small cell lung cancer cells H1299 is enhanced when combined with TAS-120 and IWR-1. H1299 cells were transfected with YFP and LLPSE-p53 plasmids for 24 hours, followed by treated with DMSO (control), TAS-120, or IWR-1 for 48 hours of continuous culture. Viability was assessed using the CCK8 assay;

FIG. 23 is a graph showing results of cell viability tests by the CCK8 kit after a buffer control or LLPSE-p53 protein were respectively delivered into a human osteosarcoma cell line U-2OS, which is then added with DMSO (control), TAS-120 or IWR-1 for continuous culture for 24 h;

FIG. 24 is a graph showing comparative phase separation results of LLPSE-p53 and its mutants in H1299 cells;

FIG. 25 is a graph showing comparative results of transactivation activity of LLPSE-p53 and other variants;

FIG. 26 is a graph showing comparative results of LLPSE-p53 and its mutants inhibiting proliferation of human tumor cell U-2OS;

FIGS. 27A-27P are diagrams showing three-dimensional structure prediction results of different phase separation enhancing sequences.

### DETAILED DESCRIPTION

In the following, design, methods and technical effect of the disclosure will be clarified with specific examples. The present disclosure includes, but is not limited to, representative embodiments disclosed below, and also can be set forth in various ways. The description is intended to help those skilled in that art comprehensively understand implementation detail of the disclosure.

In terms of DNA sequences of an p53, SEQ ID NO. 1 is a mutant p53 with normal function, and SEQ ID NO.2 is a wild-type p53. p53 in SEQ ID NO.3, SEQ ID NO.4 and SEQ ID NO.5 is human p53, p53 in SEQ ID NO.6 is p53 in hylobates moloch, p53 in SEQ ID NO.7 is p53 in rat, and p53 in SEQ ID NO.8 is p53 in naked mole rat. In other words, the sequence of p53 can be any form, it is not limited.

In the present disclosure, an amino acid sequence (including a linker) added in SEQ ID NO.3 is:

GTGGGGSGGGGSGGGGSHHHHHHGGRRRRRRRRR.

An amino acid sequence (including a linker) added in SEQ ID NO.4 is:

GGGGSHHHHHGGTGGRRRRRRR.

An amino acid sequence (including a linker) added in SEQ ID NO.5 is:

GTGGGGSGGGGSGGGGSHHHHHHHGSGGRRRRRRRRRR.

An amino acid sequence (including a linker) added in SEQ ID NO.6 is:

GGGGGHHHHHHHGGSPGGTGGKKKKKK.

An amino acid sequence (including a linker) added in SEQ ID NO.7 is:

GGASGHHHHHHHHGGAGGGGRRRRRRKK.

An amino acid sequence (including a linker) added in SEQ ID NO.8 is:

GGGSGGGSGGGSHHHHGGGDPRRRRRRRRRRRR.
SEQ ID NO. 1
ATGGAGGAGCCGCAGTCAGATCCTAGCGTCGAGCCCCCTCTGAGT
CAGGAAACATTTTCAGACCTATGGAAACTACTTCCTGAAAACAAC
GTTCTGTCCCCCTTGCCGTCCCAAGCAATGGATGATTTGATGCTG
TCCCCGGACGATATTGAACAATGGTTCACTGAAGACCCAGGTCCA
GATGAAGCTCCCAGAATGCCAGAGGCTGCTCCCCCCGTGGCCCCT
GCACCAGCAGCTCCTACACCGGCGGCCCCTGCACCAGCCCCCTCC
TGGCCCCTGTCATCTTCTGTCCCTTCCCAGAAAACCTACCAGGGC
AGCTACGGTTTCCGTCTGGGCTTCTTGCATTCTGGGACAGCCAAG
TCTGTGACTTGCACGTACTCCCCTGCCCTCAACAAGCTGTTTTGC
CAACTGGCCAAGACCTGCCCTGTGCAGCTGTGGGTTGATTCCACA
CCCCCGCCCGGCACCCGCGTCCGCGCCATGGCCATCTACAAGCAG
TCACAGCACATGACGGAGGTTGTGAGGCGCTGCCCCCACCATGAG
CGCTGCTCAGATAGCGATGGTCTGGCCCCTCCTCAGCATCTTATC
CGAGTGGAAGGAAATTTGCGTGCTGAGTATTTGGATGACAGAAAC
ACTTTTCGACATAGTGTGGTGGTGCCCTATGAGCCGCCTGAGGTT
GGCTCTGACTGTACCACCATCCACTACAACTACATGTGTTACAGT
TCCTGCATGGGCGGCATGAACCGGAGGCCCATCCTCACCATCATC
ACACTGGAAGACTCCAGTGGTAATCTACTGGGACGGGACAGCTTT
GAGGTGCGTGTTTGTGCCTGTCCTGGGAGAGACCGGCGCACAGAG
GAAGAGAATCTCCGCAAGAAAGGGGAGCCTCACCACGAGCTGCCC
CCAGGGAGCACTAAGCGAGCACTGCCCAACAACACCAGCTCCTCT
CCCCAGCCAAAGAAGAAACCACTGGATGGAGAATATTTCACCCTT
CAGATCCGTGGGCGTGAGCGCTTCGAGATGTTCCGAGAGCTGAAT
GAGGCCTTGGAACTCAAGGATGCCCAGGCTGGGAAGGAGCCAGGG
GGGAGCAGGGCTCACTCCAGCCACCTGAAGTCCAAAAAGGGTCAG
TCTACCTCCCGCCATAAAAAACTCATGTTCAAGACAGAAGGGCCT
GACTCAGAC
SEQ ID NO. 2
ATGGAGGAGCCGCAGTCAGATCCTAGCGTCGAGCCCCCTCTGAGT
CAGGAAACATTTTCAGACCTATGGAAACTACTTCCTGAAAACAAC
GTTCTGTCCCCCTTGCCGTCCCAAGCAATGGATGATTTGATGCTG
TCCCCGGACGATATTGAACAATGGTTCACTGAAGACCCAGGTCCA
GATGAAGCTCCCAGAATGCCAGAGGCTGCTCCCCCCGTGGCCCCT
GCACCAGCAGCTCCTACACCGGCGGCCCCTGCACCAGCCCCCTCC
TGGCCCCTGTCATCTTCTGTCCCTTCCCAGAAAACCTACCAGGGC
AGCTACGGTTTCCGTCTGGGCTTCTTGCATTCTGGGACAGCCAAG
TCTGTGACTTGCACGTACTCCCCTGCCCTCAACAAGATGTTTTGC
CAACTGGCCAAGACCTGCCCTGTGCAGCTGTGGGTTGATTCCACA
CCCCCGCCCGGCACCCGCGTCCGCGCCATGGCCATCTACAAGCAG
TCACAGCACATGACGGAGGTTGTGAGGCGCTGCCCCCACCATGAG
CGCTGCTCAGATAGCGATGGTCTGGCCCCTCCTCAGCATCTTATC
CGAGTGGAAGGAAATTTGCGTGTGGAGTATTTGGATGACAGAAAC
ACTTTTCGACATAGTGTGGTGGTGCCCTATGAGCCGCCTGAGGTT
GGCTCTGACTGTACCACCATCCACTACAACTACATGTGTAACAGT
TCCTGCATGGGCGGCATGAACCGGAGGCCCATCCTCACCATCATC
ACACTGGAAGACTCCAGTGGTAATCTACTGGGACGGAACAGCTTT
GAGGTGCGTGTTTGTGCCTGTCCTGGGAGAGACCGGCGCACAGAG
GAAGAGAATCTCCGCAAGAAAGGGGAGCCTCACCACGAGCTGCCC
CCAGGGAGCACTAAGCGAGCACTGCCCAACAACACCAGCTCCTCT
CCCCAGCCAAAGAAGAAACCACTGGATGGAGAATATTTCACCCTT
CAGATCCGTGGGCGTGAGCGCTTCGAGATGTTCCGAGAGCTGAAT
GAGGCCTTGGAACTCAAGGATGCCCAGGCTGGGAAGGAGCCAGGG
GGGAGCAGGGCTCACTCCAGCCACCTGAAGTCCAAAAAGGGTCAG
TCTACCTCCCGCCATAAAAAACTCATGTTCAAGACAGAAGGGCCT
GACTCAGAC
SEQ ID NO. 3
ATGGAGGAGCCGCAGTCAGATCCTAGCGTCGAGCCCCCTCTGAGT
CAGGAAACATTTTCAGACCTATGGAAACTACTTCCTGAAAACAAC
GTTCTGTCCCCCTTGCCGTCCCAAGCAATGGATGATTTGATGCTG
TCCCCGGACGATATTGAACAATGGTTCACTGAAGACCCAGGTCCA
GATGAAGCTCCCAGAATGCCAGAGGCTGCTCCCCCCGTGGCCCCT
GCACCAGCAGCTCCTACACCGGCGGCCCCTGCACCAGCCCCCTCC
TGGCCCCTGTCATCTTCTGTCCCTTCCCAGAAAACCTACCAGGGC
AGCTACGGTTTCCGTCTGGGCTTCTTGCATTCTGGGACAGCCAAG
TCTGTGACTTGCACGTACTCCCCTGCCCTCAACAAGCTGTTTTGC
CAACTGGCCAAGACCTGCCCTGTGCAGCTGTGGGTTGATTCCACA
CCCCCGCCCGGCACCCGCGTCCGCGCCATGGCCATCTACAAGCAG
TCACAGCACATGACGGAGGTTGTGAGGCGCTGCCCCCACCATGAG
CGCTGCTCAGATAGCGATGGTCTGGCCCCTCCTCAGCATCTTATC
CGAGTGGAAGGAAATTTGCGTGCTGAGTATTTGGATGACAGAAAC
ACTTTTCGACATAGTGTGGTGGTGCCCTATGAGCCGCCTGAGGTT
GGCTCTGACTGTACCACCATCCACTACAACTACATGTGTTACAGT
TCCTGCATGGGCGGCATGAACCGGAGGCCCATCCTCACCATCATC
ACACTGGAAGACTCCAGTGGTAATCTACTGGGACGGGACAGCTTT
GAGGTGCGTGTTTGTGCCTGTCCTGGGAGAGACCGGCGCACAGAG
GAAGAGAATCTCCGCAAGAAAGGGGAGCCTCACCACGAGCTGCCC
CCAGGGAGCACTAAGCGAGCACTGCCCAACAACACCAGCTCCTCT
CCCCAGCCAAAGAAGAAACCACTGGATGGAGAATATTTCACCCTT
CAGATCCGTGGGCGTGAGCGCTTCGAGATGTTCCGAGAGCTGAAT
GAGGCCTTGGAACTCAAGGATGCCCAGGCTGGGAAGGAGCCAGGG
GGGAGCAGGGCTCACTCCAGCCACCTGAAGTCCAAAAAGGGTCAG
TCTACCTCCCGCCATAAAAAACTCATGTTCAAGACAGAAGGGCCT
GACTCAGACGGTACCGGTGGAGGAGGTTCTGGAGGCGGTGGAAGT
GGTGGCGGAGGTAGCCACCACCACCACCACCACGGTGGTCGTCGT
AGACGTCGTCGTCGACGTCGT
SEQ ID NO. 4
ATGGAGGAGCCGCAGTCAGATCCTAGCGTCGAGCCCCCTCTGAGT
CAGGAAACATTTTCAGACCTATGGAAACTACTTCCTGAAAACAAC
GTTCTGTCCCCCTTGCCGTCCCAAGCAATGGATGATTTGATGCTG
TCCCCGGACGATATTGAACAATGGTTCACTGAAGACCCAGGTCCA
GATGAAGCTCCCAGAATGCCAGAGGCTGCTCCCCCCGTGGCCCCT
GCACCAGCAGCTCCTACACCGGCGGCCCCTGCACCAGCCCCCTCC
TGGCCCCTGTCATCTTCTGTCCCTTCCCAGAAAACCTACCAGGGC
AGCTACGGTTTCCGTCTGGGCTTCTTGCATTCTGGGACAGCCAAG
TCTGTGACTTGCACGTACTCCCCTGCCCTCAACAAGCTGTTTTGC
CAACTGGCCAAGACCTGCCCTGTGCAGCTGTGGGTTGATTCCACA
CCCCCGCCCGGCACCCGCGTCCGCGCCATGGCCATCTACAAGCAG
TCACAGCACATGACGGAGGTTGTGAGGCGCTGCCCCCACCATGAG
CGCTGCTCAGATAGCGATGGTCTGGCCCCTCCTCAGCATCTTATC
CGAGTGGAAGGAAATTTGCGTGCTGAGTATTTGGATGACAGAAAC
ACTTTTCGACATAGTGTGGTGGTGCCCTATGAGCCGCCTGAGGTT
GGCTCTGACTGTACCACCATCCACTACAACTACATGTGTTACAGT
TCCTGCATGGGCGGCATGAACCGGAGGCCCATCCTCACCATCATC
ACACTGGAAGACTCCAGTGGTAATCTACTGGGACGGGACAGCTTT
GAGGTGCGTGTTTGTGCCTGTCCTGGGAGAGACCGGCGCACAGAG
GAAGAGAATCTCCGCAAGAAAGGGGAGCCTCACCACGAGCTGCCC
CCAGGGAGCACTAAGCGAGCACTGCCCAACAACACCAGCTCCTCT
CCCCAGCCAAAGAAGAAACCACTGGATGGAGAATATTTCACCCTT
CAGATCCGTGGGCGTGAGCGCTTCGAGATGTTCCGAGAGCTGAAT
GAGGCCTTGGAACTCAAGGATGCCCAGGCTGGGAAGGAGCCAGGG
GGGAGCAGGGCTCACTCCAGCCACCTGAAGTCCAAAAAGGGTCAG
TCTACCTCCCGCCATAAAAAACTCATGTTCAAGACAGAAGGGCCT
GACTCAGACGGTGGCGGAGGTAGCCACCACCACCACCACGGCGGT
ACCGGTGGTCGTCGTAGACGTCGTCGTCGA
SEQ ID NO. 5
ATGGAGGAGCCGCAGTCAGATCCTAGCGTCGAGCCCCCTCTGAGT
CAGGAAACATTTTCAGACCTATGGAAACTACTTCCTGAAAACAAC
GTTCTGTCCCCCTTGCCGTCCCAAGCAATGGATGATTTGATGCTG
TCCCCGGACGATATTGAACAATGGTTCACTGAAGACCCAGGTCCA
GATGAAGCTCCCAGAATGCCAGAGGCTGCTCCCCCCGTGGCCCCT
GCACCAGCAGCTCCTACACCGGCGGCCCCTGCACCAGCCCCCTCC
TGGCCCCTGTCATCTTCTGTCCCTTCCCAGAAAACCTACCAGGGC
AGCTACGGTTTCCGTCTGGGCTTCTTGCATTCTGGGACAGCCAAG
TCTGTGACTTGCACGTACTCCCCTGCCCTCAACAAGATGTTTTGC
CAACTGGCCAAGACCTGCCCTGTGCAGCTGTGGGTTGATTCCACA
CCCCCGCCCGGCACCCGCGTCCGCGCCATGGCCATCTACAAGCAG
TCACAGCACATGACGGAGGTTGTGAGGCGCTGCCCCCACCATGAG
CGCTGCTCAGATAGCGATGGTCTGGCCCCTCCTCAGCATCTTATC
CGAGTGGAAGGAAATTTGCGTGTGGAGTATTTGGATGACAGAAAC
ACTTTTCGACATAGTGTGGTGGTGCCCTATGAGCCGCCTGAGGTT
GGCTCTGACTGTACCACCATCCACTACAACTACATGTGTAACAGT
TCCTGCATGGGCGGCATGAACCGGAGGCCCATCCTCACCATCATC
ACACTGGAAGACTCCAGTGGTAATCTACTGGGACGGAACAGCTTT
GAGGTGCGTGTTTGTGCCTGTCCTGGGAGAGACCGGCGCACAGAG
GAAGAGAATCTCCGCAAGAAAGGGGAGCCTCACCACGAGCTGCCC
CCAGGGAGCACTAAGCGAGCACTGCCCAACAACACCAGCTCCTCT
CCCCAGCCAAAGAAGAAACCACTGGATGGAGAATATTTCACCCTT
CAGATCCGTGGGCGTGAGCGCTTCGAGATGTTCCGAGAGCTGAAT
GAGGCCTTGGAACTCAAGGATGCCCAGGCTGGGAAGGAGCCAGGG
GGGAGCAGGGCTCACTCCAGCCACCTGAAGTCCAAAAAGGGTCAG
TCTACCTCCCGCCATAAAAAACTCATGTTCAAGACAGAAGGGCCT
GACTCAGACggtACCGGTGGAGGAGGTTCTGGAGGCGGTGGAAGT
GGTGGCGGAGGTAGCCACCACCACCACCACCACCACGGTAGTGGT
GGCCGTCGTAGACGTCGTCGTCGACGTCGTCGT
SEQ ID NO. 6
ATGGAGGAGCCGCAGTCAGATCCTAGCGTCGAGCCCCCTCTGAGT
CAGGAAACATTTTCAGACCTATGGAAACTACTTCCTGAAAACAAC
GTTCTGTCCCCCTTGCCGTCCCAAGCGATGGATGATTTGATGCTG
TCCCCGGAAGATATTGCACAATGGTTCACTGAAGACCCAGGTCCA
CATGAAGCTCCCAGAATGTCAGAGGCTGCTCCCCCCATGGCCCCC
GCACCAGGAGCTCCTACACTGGCGGCGCCTGCACCAGCCCCCTCC
TGGCCCCTGTCATCCTCTGTCCCTTCCCAGAAAACCTACCAGGGC
AGCTACGGTTTCCGTCTGGGCTTCTTGCATTCTGGAACGGCCAAG
TCTGTGACTTGCACGTACTCCCCTGCCCTCAACAAGATGTTTTGC
CAGCTGGCCAAGACCTGCCCTGTGCAGCTGTGGGTTGATTCCACA
CCCCCTCCCGGCACCCGTGTCCGCGCCATGGCCATCTACAAGCAG
TCACAGCACATGACGGAGGTTGTGAGGCGCTGCCCCCACCATGAG
CGCTGCTCAGATAGCGATGGTCTGGCCCCTCCTCAGCATCTTATC
CGAGTGGAAGGCAATTTGCGTGTGGAGTATTTGGATGACAGAAAC
ACTTTTCGACATAGTGTGGTGGTGCCCTATGAGCCGCCTGAGGTT
GGCTCTGACTGTACCACCATCCACTACAACTACATGTGTAACAGT
TCCTGCATGGGCGGCATGAACCGGAGGCCCATCCTCACCATCATC
ACACTGGAAGACTCCAGTGGTAATCTACTGGGACGGAACAGCTTT
GAGGTGCGCGTTTGTGCCTGTCCTGGGAGAGACCGGCGCACAGAG
GAAGAGAATTTCCACAAGAAAGGGGAGCCTCACCACGAGCTGCCC
CCTGGGAGCACTAAGCGAGCACTGCCCAACAACACCAGCTCCTCT
CCCCAGCCAAAGAAGAAACCACTGGATGGAGAATATTTCACCCTT
CAGATCCGTGGGCGTGAGCGCTTCGAGATGTTCCGAGAGCTGAAT
GAGGCTTTGGAACTCAAGGATGCCCAGGCTGGGAAGGAGCCAGGG
GGGAGCAGGGCTCACTCCAGCCACCTGAAGTCCAAGAAGGGTCAG
TCTACCTCCCGCCATAAAAAACTTATGTTCAAGACAGAAGGGCCT
GACTCAGACGGCGGTGGCGGCGGCCACCACCACCACCACCACCAC
GGCGGCAGCCCCGGCGGCACCGGCGGCAAGAAGAAGAAGAAGAAG
SEQ ID NO. 7
ATGGAGGATTCACAGTCGGATATGAGCATCGAGCTCCCTCTGAGT
CAGGAGACATTTTCATGCTTATGGAAACTTCTTCCTCCAGATGAT
ATTCTGCCCACCACAGCGACAGGGTCACCTAATTCCATGGAAGAT
CTGTTCCTGCCCCAGGATGTTGCAGAGTTGTTAGAAGGCCCAGAG
GAAGCCCTCCAAGTGTCAGCTCCTGCAGCACAGGAACCTGGAACT
GAGGCCCCTGCACCCGTGGCCCCTGCTTCAGCTACACCGTGGCCT
CTGTCATCTTCCGTCCCTTCTCAAAAAACTTACCAAGGCAACTAT
GGCTTCCACCTGGGCTTCCTGCAGTCAGGGACAGCCAAGTCTGTT
ATGTGCACGTACTCAATTTCCCTCAATAAGCTGTTCTGCCAGCTG
GCGAAGACATGCCCTGTGCAGTTGTGGGTCACCTCCACACCTCCA
CCTGGTACCCGTGTCCGTGCCATGGCCATCTACAAGAAGTCACAA
CACATGACTGAGGTCGTGAGACGCTGCCCCCACCATGAGCGTTGC
TCTGATGGTGACGGCCTGGCTCCTCCCCAACATCTTATCCGGGTG
GAAGGAAATCCGTATGCTGAGTATCTGGACGACAGGCAGACTTTT
CGGCACAGCGTGGTGGTACCGTATGAGCCACCTGAGGTCGGCTCC
GACTATACCACTATCCACTACAAGTACATGTGCAACAGCTCCTGC
ATGGGGGGCATGAACCGCCGGCCCATCCTTACCATCATCACGCTG
GAAGACTCCAGTGGGAATCTTCTGGGACGGGACAGCTTTGAGGTT
CGTGTTTGTGCCTGTCCTGGGAGAGACCGTCGGACAGAGGAAGAA
AATTTCCGCAAAAAAGAAGAGCATTGCCCGGAGCTGCCCCCAGGG
AGTGCAAAGAGAGCACTGCCCACCAGCACAAGCTCCTCTCCCCAG
CAAAAGAAAAAACCACTCGATGGAGAATATTTCACCCTTAAGATC
CGTGGGCGTGAGCGCTTCGAGATGTTCCGAGAGCTGAATGAGGCC
TTGGAATTAAAGGATGCCCGTGCTGCCGAGGAGTCAGGAGACAGC
AGGGCTCACTCCAGCTACCCGAAGACCAAGAAGGGCCAGTCTACG
TCCCGCCATAAAAAACCAATGATCAAGAAAGTGGGGCCTGACTCA
GACGGAGGCGCAAGTGGTCACCACCACCACCACCACCACCACGGC
GGCGCCGGCGGCGGCGGCAGGAGGAGGAGGAGGAGGAAGAAG
SEQ ID NO. 8
ATGTACCCTCCCCTAGCAGGTCCTCTGCCTAGCTCTCTGACTAGG
GTTGGCTATATCTCACACCCAGTCCTCATTTTTCCCCTCATAGCA
GCCTTCTTAGCTGCTGCCATGGAAGAGCCACAGTCGGATCTCAGC
ATCGAGCCTCCACTGAGTCAGGAGACATTTTCAGACTTATGGAAA
CTACTTCCTGAAAACAACGTTCTGTCCAGCTCACTGTCCTCTCCC
ATGGATGATCTGCTGCTGTCCCCAGAAGATGTTGTAAACTGGCTG
GGAGGAAACCCAGATGAAGATGTCCAAGTGTCAGCAGCTCCTGTA
CCAGAGCCCCCAACACCAGTGGCCCCTGCCCCGGCAGCTCCCGCA
CCAGCCACTTCCTGGCCTCTGTCATCCTCCGTCCCTTCCCATAAG
ACCTACCAAGGCAACTATGGTTTCCATCTGGGCTTCCTTCAGTCT
GGGACGGCCAAATCTGTCACATGCACGTACTCCCCTGTTCTCAAC
AAGTTATTCTGCCAACTGGCAAAGACCTGCCCTGTGCAAGTGTGG
GTCGAATCACCACCCCCACCTGGCACCCGAGTCCGTGCCATGGCC
ATCTACAAGAAGTCACAGCACATGACAGAAGTTGTGAGGCGCTGC
CCCCACCATGAGCGCTGCTCCGATAGTGATGGCCTGGCCCCTCCT
CAGCATCTTATCCGGGTGGAAGGAAATCTGCGTGCAGAATATTTG
GATGACAGAACCACTTTTCGCCATAGCGTGGTGGTACCCTATGAT
CTGCCTGAGGTTGGCTCTGACTGTACCACCATCCACTACAACTAT
ATGTGCAACAGTTCTTGCATGGGGGGCATGAACCGTAGGCCCATC
CTCACCATTATCACACTGGAAGACTCCAGTGGGAACCTGCTGGGG
CGGAACAGCTTTGAGGTGCGTGTTTGTGCCTGTCCTGGGAGAGAC
CGGCGCACAGAGGAAGAAAATTTCCACAAGAAAGGGGGGTCATGC
CCAGAGCCAACACCAGGAAGCATTAAGCGAGCACTGCCCACTGGC
ACCAACTCTTCTCCTCAGCCAAAGAAGAAACCACTGGATGGGGAA
TATTTCACCCTTAAGATCCGTGGGCGTGAACGCTTTGAGATGTTC
CGAGAGCTAAATGAGGCCTTGGAACTCAAGGATGCCCAAACTGAG
AAGGAGCCAGGGGAGAGCAGGCCTCACTCAAGCTACCTGAAGTCT
AAGAAGGGGCAGTCTACCTCCTGTCATAAAAAACTAATGTTCAAG
AAAGAAGGACCTGATTCAGACGGAGGGGGTTCCGGTGGCGGTTCC
GGTGGCGGTTCTCACCACCACCACGGTGGCGGGGATCCACGTCGT
AGACGTCGTCGTCGACGTCGTAGACGTCGT.

### EXAMPLE 1 COMPARISON OF IN-VITRO PHASE SEPARATION ABILITY BETWEEN WT-p53 AND EGFP-p53 RECOMBINANT PROTEINS IN VITRO

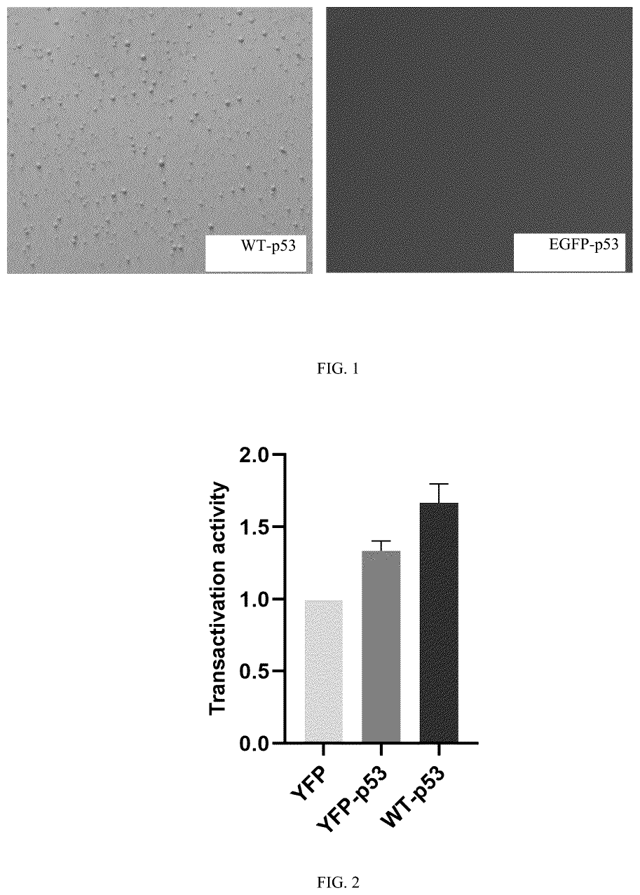

Because tags can affect a structure of protein, in order to determine whether liquid-liquid phase separation ability of p53 can be enhanced by randomly adding tags, EGFP was added to a C-terminus of p53, and then liquid-liquid phase separation ability of WT-p53 (without fluorescent tag proteins) and EGFP-p53 recombinant proteins was compared in vitro. Experimental results show that, under identical conditions, unlike WT-p53 without a fluorescent tag, which undergoes phase separation forming visible droplets, EGFP-p53 does not form discernible droplets, indicating that the EGFP tag has obvious inhibitory effect on liquid-liquid phase separation of the p53 protein (FIG. 1).

Implementation was made as follows:
1. EGFP-p53 and WT-p53 were separately sub-cloned into pET-24a (+) prokaryotic expression vectors, which were transformed into a C41 E.coli expression strain, and the C41 cells were then cultured overnight in a constant temperature incubator at 37° C.
2. The monoclone was picked out, cultured at 37° C. and 220 rpm until OD600=0.6.
3. 200 to 500 μM of IPTG (Isopropyl β-d-1-thiogalactopyranoside) and 100 μM of ZnCl₂ were added, the cells were cultured overnight at 25° C., 220 rpm.
4. Centrifugation was conducted at 4,500 rpm for 30 min at 4° C.
5. Cell lysate was added, crushed by high-pressure homogenizer, and centrifuged at 17000 rpm at 4° C. for 30 min;
6. The supernatant was collected and purified with Histrap™ HP resin.
7. Eluent was purified with HiTrap™ Heparin HP resin.
8. Finally, Gel filtration was carried out for further purification.
9. Phase separation systems were prepared, which were imaged under a fluorescence inverted microscope, and their phase separation ability were compared.

Results show that under the same conditions, obvious droplets have been formed for WT-p53, but not for EGFP-p53, indicating that EGFP, a tag protein, does not promote but inhibit liquid-liquid phase separation of p53 protein.

### EXAMPLE 2 COMPARISON OF TRANSACTIVATION ACTIVITY BETWEEN YFP-p53 AND WT-p53

In order to investigate whether transactivation activity of p53 is changed by adding any tag to one terminus of p53, a YFP (Yellow Fluorescent Protein) tag was added to the C-terminus of wild-type p53 for constructing a eukaryotic expression vector of pCMV3-YFP-p53, and then pCMV3-YFP-p53 and pCMV3-p53 were transfected into cells, and their effects on the transactivation activity of CDKN1A were tested. Experimental results show that compared with WT-p53, transcriptional activation ability of YFP-p53 on CDKN1A tends to decrease (FIG. 2).

Implementation was made as follows:
1. Proper number of cells were seeded into a 96-well cell culture plate, plasmids and CDKN1A reporter gene plasmid were co-transformed when cell density reached 60-70%, the cells were then cultured in an incubator for 24 h.
2. A chemiluminescence test was carried out with a luciferase reporting system test kit produced by Promega and a M5 microplate reader.
3. Graphpad5 software was used for data processing and analysis.

Results show that compared with WT-p53, YFP-p53 had weaker transactivation activity on CDKN1A, indicating that adding the YFP tag does not promote but instead inhibits the transactivation activity of wild-type p53.

### EXAMPLE 3 COMPARISON OF LIQUID-LIQUID PHASE SEPARATION ABILITY OF WT-p53 AND LLPSE-p53 (SEQ ID NO.3) IN CELLS OF HUMAN RENAL EPITHELIAL CELL LINE HEK293T

In order to verify that phase separation ability of a p53 variant LLPSE-p53 designed in this disclosure is better than that of WT-p53, eukaryotic expression vectors of pEGFP-p53 and pEGFP-LLPSE-p53 were constructed, and numbers and sizes of droplets formed in cells by them were measured by transfecting to human embryonic kidney cells HEK293T, and investigated by immunofluorescence technology.

Implementation was made as follows:
1. 6×10⁵ to 8×10⁵ HEK293T cells were seeded in per 35 mm confocal dish one day before transfection, and 2 ml of complete medium was added.
2. After cell density reached 60-70%, 2 to 2.5 μg of plasmid were transfected into each well, the dishes were then cultured for 24 h in a 5% CO₂ incubator at 37° C.
3. 1 ml of complete medium was sucked and discarded, 1 ml of 4% paraformaldehyde was added and then fixed at room temperature for 10 min.
4. All supernatant was sucked and discarded, and 1 ml of PBS (phosphate buffered saline) was added and rinsed for 3 times, 5 min each time.
5. 0.2% of Triton™ X-100 was added, the cells were permeated at room temperature for 15 min.
6. All supernatant was sucked and discarded, and 1 ml of PBS was added and rinsed for 3 times, 5 min each time.
7. 1 ml of 5% BSA (bovine serum albumin) was added and sealed at room temperature for 1 h.
8. Diluted p53 (DO-1) primary antibody (with a dilution ratio of 1:1000) was added, the dishes were then placed on a horizontal shaker and incubated at 4° C. overnight.
9. The primary antibody was recovered, and 1 ml of PBS was added and rinsed for 3 times, 5 min each time.
10. Diluted immunofluorescence secondary antibody (with a dilution ratio of 1:1000) was added and incubated for 1 h at room temperature in the dark.
11. Secondary antibody was sucked and discarded, and 1 ml of PBS was added and rinsed for 3 times, 5 min each time.
12. A confocal microscope was used to take photos for measurement, with consistent parameters for different samples.

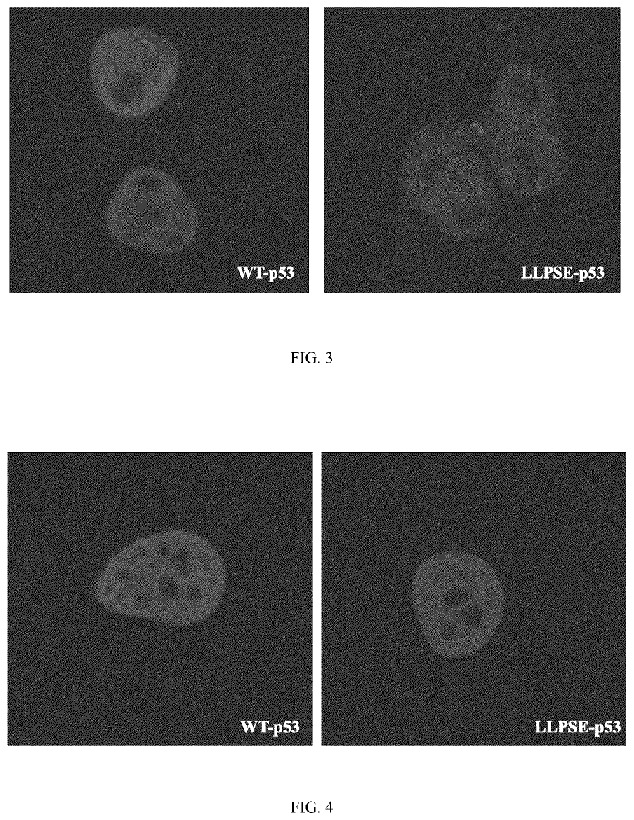

Experimental results show that compared with WT-p53, LLPSE-p53 forms larger and more droplets in HEK293T cells (FIG. 3).

### EXAMPLE 4 COMPARISON OF LIQUID-LIQUID PHASE SEPARATION OF WT-p53 AND LLPSE-p53 (SEQ ID NO.4) IN CELLS OF HUMAN NON-SMALL CELL LUNG CANCER CELL LINE H1299

In order to verify that phase separation ability of a p53 variant LLPSE-p53 designed in this disclosure is better than that of WT-p53, eukaryotic expression vectors of pCMV6-p53 and pCMV6-LLPSE-p53 were constructed, and numbers and sizes of droplets formed in cells by them were measured using immunofluorescence technology after above plasmids were transfected to a human non-small cell lung cancer cell line H1299.

Implementation was made as follows:

Specific implementation steps can refer to Example 3. The HEK293T cells in step 1 in Example 3 were replaced with H1299 cells.

Experimental results show that compared with WT-p53, LLPSE-p53 forms larger and more droplets in H1299 cells (FIG. 4).

### EXAMPLE 5 COMPARISON OF LIQUID-LIQUID PHASE SEPARATION ABILITY OF WT-p53 (NAKED MOLE RAT) AND LLPSE-p53 (NAKED MOLE RAT, SEQ ID NO.8) IN CELLS OF HUMAN NON-SMALL CELL LUNG CANCER CELL LINE H1299

In order to verify whether constructed p53 sequences with enhanced phase separation ability in the disclosure can also be applied to p53 of other species, a sequence fragment with enhanced phase separation was added to p53 gene (NMR-p53) of naked mole rat, eukaryotic expression vectors of pCMV6-p53(NMR) and pCMV6-LLPSE-p53(NMR) were constructed and transfected into the human non-small cell lung cancer cell line H1299, and numbers and sizes of droplets formed in cells by them were measured and compared by taking photos with a confocal microscope.

Implementation was made as follows:

Specific implementation steps can refer to Example 3. The HEK293T cells in step 1 in Example 3 were replaced with H1299 cells.

Experimental results show that compared with WT-p53 (NMR), LLPSE-p53 (NMR) forms larger and more droplets in H1299 cells (FIG. 5).

### EXAMPLE 6 p53 VARIANT OF THE PRESENT DISCLOSURE (SEQ ID NO.5) HAS IMPROVED ABILITY OF TRANSCRIPTIONAL ACTIVATION OF ITS DOWNSTREAM TARGET GENES

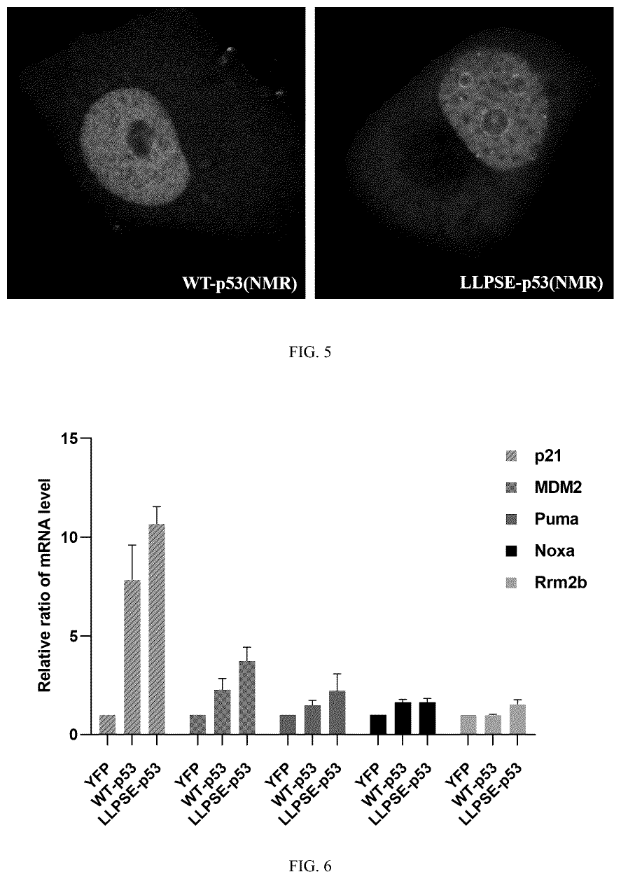

After it is proved that the liquid-liquid phase separation ability of LLPSE-p53 is better than that of WT-p53, eukaryotic expression vectors of pEGFP-p53 and pEGFP-LLPSE-p53 were constructed, and transcriptional activation activity of WT-p53 and LLPSE-p53 on p53 downstream target genes was measured by qRT-PCR assay in HEK293T cells. Results show that compared with WT-p53, LLPSE-p53 significantly improves transcription levels of CDKN1A, MDM2 and PUMA (FIG. 6). It indicates that liquid-liquid phase separation enhances transcriptional activation activity of p53.

Implementation was made as follows:
1. Cells were seeded into a 12-well cell culture plate, 1 μg of plasmid was transfected after cell density reached 70-80%, the cells were then cultured for 24 h.
2. Total RNA was extracted using Trizol.
3. The extracted RNA was reverse transcribed and synthesized into cDNA, and a qRT-PCR system was made according to a standardized process.
4. Measurement was made using a Real-time fluorescence quantitative PCR instrument.
5. Graphpad5 software was used for data processing and analysis.

Experimental results show that LLPSE-p53 has stronger transactivation activity than WT-p53.

### EXAMPLE 7 p53 VARIANT OF THE PRESENT DISCLOSURE (SEQ ID NO.6) HAS IMPROVED ABILITY OF TRANSCRIPTIONAL ACTIVATION OF ITS DOWNSTREAM TARGET GENES

It has been demonstrated that phase separation could enhance the transcriptional activity of p53 in HEK293T cells above. In Example 7, the eukaryotic expression vectors of pCMV6-p53 and pCMV6-LLPSE-p53 were constructed, for further verification in H1299 cells. Experimental results show that compared with WT-p53, LLPSE-p53 significantly improves transcription levels of CDKN1A, MDM2 and PUMA (FIG. 7).

Implementation was made as follows:

Specific implementation steps can refer to Example 6.

Experimental results show that LLPSE-p53 has stronger transactivation activity than WT-p53.

### EXAMPLE 8 p53 VARIANT OF THE PRESENT DISCLOSURE (SEQ ID NO.7) HAS IMPROVED TRANSACTIVATION ACTIVITY

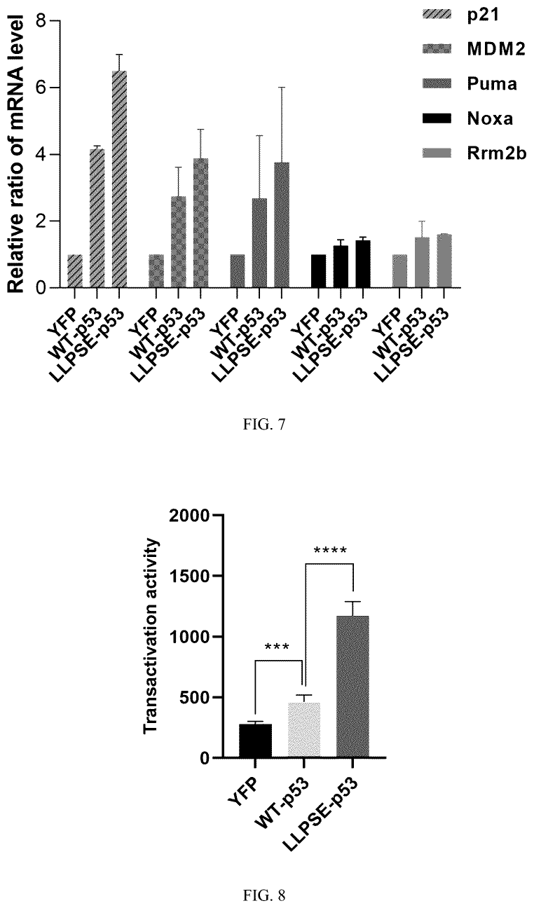

In order to further investigate whether the transactivation activity of LLPSE-p53 is better than that of WT-p53, pcDNA3.1-p53 and pcDNA3.1-LLPSE-p53 eukaryotic expression vectors were constructed, and it is proven that LLPSE-p53 can promote the transcriptional activation of CDKN1A in H1299 cells by luciferase report experiments (FIG. 8).

Implementation was made as follows:
1. Proper number of cells were seeded into a 96-well cell culture plate, plasmids and CDKN1A reporter gene plasmid were co-transformed when cell density reached 60-70%, the cells were then cultured in an incubator for 24 h.
2. A chemiluminescence assay was carried out with the luciferase reporting system kit produced by Promega and a M5 microplate reader.
3. Graphpad5 software was used for data processing and analysis.

Results show that compared with WT-p53, LLPSE-p53 significantly increases the transactivation activity of CDKN1A.

### EXAMPLE 9 p53 VARIANT OF THE PRESENT DISCLOSURE (SEQ ID NO.3) HAS IMPROVED ABILITY TO KILL NON-SMALL CELL LUNG CANCER CELL LINE H1299

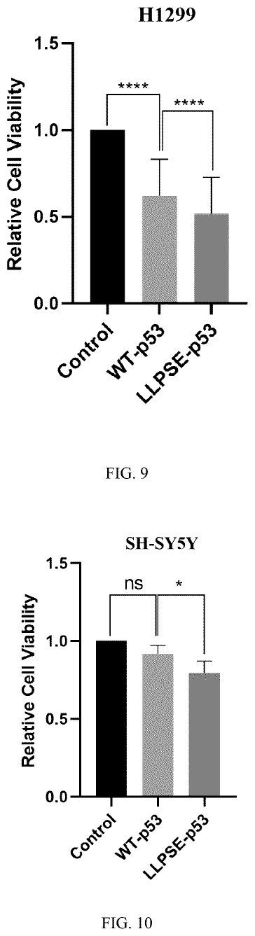

Having proved that liquid-liquid phase separation improves the transcriptional activation ability of p53 above, here we further investigated whether phase separation can enhance its killing effect on tumors. Eukaryotic expression vectors of pEGFP-p53 and pEGFP-LLPSE-p53 were constructed, and cell viability was measured by a plasmids transfection and ATP method. The data show that the p53 variant with improved liquid-liquid phase separation ability significantly inhibits proliferation of H1299 cells, and LLPSE-p53 can significantly inhibit the proliferation of H1299 tumor cells compared with WT-p53 (FIG. 9).

Implementation was made as follows:
1. 5000 cells/well were seeded into a 96-well cell culture plate, 100 μl of complete medium was added to each well, the cells were then placed in an incubator for continuous culture for 12-24 h.
2. 100 ng to 200 ng of plasmid was transfected into cells, with 3 replicates in each group, the plate was then placed in an incubator for continuous culture for 72 h.
3. The cells were taken out and equilibrated at room temperature for 30 min, and the working solution was also equilibrated to room temperature.
4. 50 μl of complete medium was sucked and discarded, 50 μl of working solution was added, which was then placed in a horizontal shaker for 15 min at room temperature.
5. Chemiluminescence measurement was carried out for 1-3 s on each well by using a M5 microplate reader.
6. Graphpad5 software was used for data processing and analysis.

Results show that compared with WT-p53, LLPSE-p53 can significantly inhibit proliferation of H1299 tumor cells.

### EXAMPLE 10 p53 VARIANT OF THE PRESENT DISCLOSURE (SEQ ID NO.3) HAS IMPROVED ABILITY TO KILL NEUROBLASTOMA CELL LINE SH-SY5Y

In order to explore whether LLPSE-p53 has improved killing effect on other tumors, eukaryotic expression vectors of pCMV3-p53 and pCMV3-LLPSE-p53 were constructed and transfected into SH-SY5Y cells, and cell viability was measured. The data show that the p53 variant with improved liquid-liquid phase separation ability significantly inhibits proliferation of SH-SY5Y cells, with improved effect over WT-p53 (FIG. 10).

Implementation was made as follows:

Specific implementation steps can refer to Example 9.

Results show that compared with WT-p53, LLPSE-p53 has improved killing effect on SH-SY5Y cells.

### EXAMPLE 11 p53 VARIANT OF THE PRESENT DISCLOSURE (SEQ ID NO.3) HAS IMPROVED ABILITY TO INHIBIT PROLIFERATION OF OSTEOSARCOMA CELL LINE U-2OS

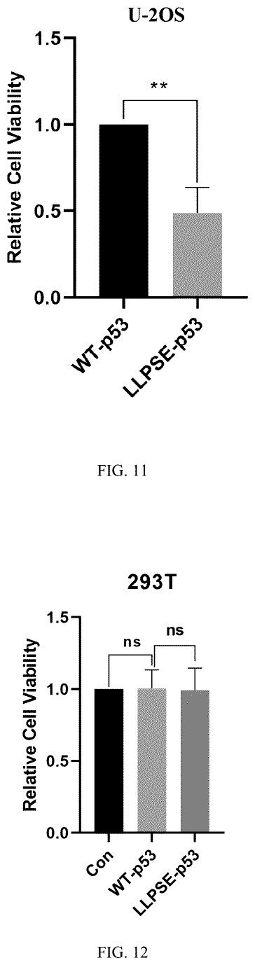

In order to further explore killing effect of LLPSE-p53 on tumor, eukaryotic expression vectors of pcDNA6B-p53 and pcDNA6B-LLPSE-p53 were constructed, and cell viability was also measured in U-2OS cells. Experimental results show that the p53 variant with improved liquid-liquid phase separation ability significantly inhibits proliferation of U-2OS tumor cells, with improved effect over WT-p53 (FIG. 11).

Implementation was made as follows:

Specific implementation steps can refer to Example 9. Results show that compared with WT-p53, LLPSE-p53 has significant higher killing effect on the U-2OS tumor cells.

### EXAMPLE 12 THE p53 VARIANT OF THE PRESENT DISCLOSURE (SEQ ID NO.3) HAS NO EFFECT ON PROLIFERATION OF NORMAL HUMAN EMBRYONIC KIDNEY CELLS

In order to explore whether LLPSE-p53 of the present disclosure causes toxicity to human normal cell lines, eukaryotic expression vectors of pcDNA6B-p53 and pcDNA6B-LLPSE-p53 were constructed, and cell viability was measured after plasmids were transfected into human embryonic kidney cell line HEK293T. Experimental data show that LLPSE-p53 may not affect cell viability of HEK293T (FIG. 12).

Implementation was made as follows:
1. 5000 cells/well were seeded into a 96-well cell culture plate, 100 μl of complete medium was added to each well, the plate was then placed in an incubator for continuous culture for 12-24 h.
2. 100 ng to 200 ng of plasmid was transfected into cells, with 3 wells in each group, the plate was then placed in an incubator for 72 h.
3. Cell proliferation was measured by a CCK8 method.
4. Graphpad5 software was used for data processing and analysis.

Results show that compared with WT-p53, LLPSE-p53 is not toxic to the human normal cell line HEK293T.

### EXAMPLE 13 COMPARISON OF LIQUID-LIQUID PHASE SEPARATION ABILITY BETWEEN p53 VARIANT (SEQ ID NO.3) RECOMBINANT PROTEIN OF THE PRESENT DISCLOSURE AND WT-p53 RECOMBINANT PROTEIN IN VITRO

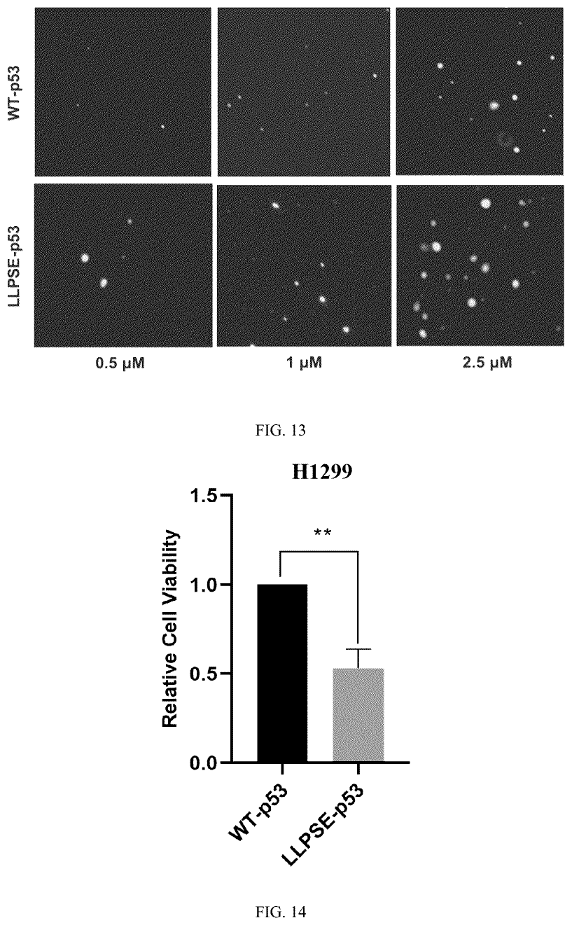

After clarifying the phase separation ability and function of LLPSE-p53 at the expression level in eukaryotic cells above, we further compared the phase separation ability of LLPSE-p53 and WT-p53 recombinant proteins in vitro. Phase separation experiments in vitro were carried out after prokaryotic expression and purification of WT-p53 and LLPSE-p53 recombinant proteins. Phase separation experiments on WT-p53 and LLPSE-p53 recombinant proteins showed that the phase separation capability of LLPSE-p53 is much stronger than that of WT-p53 (FIG. 13).

Implementation was made as follows:

Specific implementation steps can refer to Example 1. The difference from Example 1 involves performing transformation into a BL21 E. coli expression strain in Step 1.

Results show that more and larger droplets are formed for LLPSE-p53 than for WT-p53, indicating that LLPSE-p53 has stronger liquid-liquid phase separation ability than WT-p53.

### EXAMPLE 14 p53 VARIANT (SEQ ID NO.3) PROTEIN OF THE PRESENT DISCLOSURE HAS IMPROVED ABILITY TO KILL HUMAN NON-SMALL CELL LUNG CANCER CELL LINE H1299

After it was proven that phase separation ability of LLPSE-p53 recombinant protein is better than that of WT-p53 in vitro above, in Example 14 it was further found that LLPSE-p53 protein had a stronger killing effect on H1299 tumor cells compared with WT-p53 recombinant protein through protein delivery into cells experiments (FIG. 14).

Implementation was made as follows:
1. 5000 cells/well were seeded into a 96-well cell culture plate, 100 μl of complete medium was added (with no P/S), the plate was then placed in an incubator for 12-24 h.
2. 50 μl of protein delivery system was prepared according to a protein final concentration of 6.5-10 μM as follows:
6.5-10 μM of protein+5×transduction buffer+complete medium (no P/S).
3. The above system was sterilized with a 0.22 μm filter membrane.
4. The complete medium in the well plate was sucked and discarded, and the above protein delivery system was added, the plate was then placed in an incubator for 24 h.
5. The protein delivery system was sucked and discarded, and complete medium was added, the plate was then cultured for another 24 h.
6. Cell viability was measured by an ATP method and with a M5 microplate reader.
7. Graphpad5 software was used for data processing and analysis.

Results show that for H1299 tumor cells, LLPSE-p53 recombinant protein has a stronger tumor killing effect on H1299 tumor cells compared with WT-p53.

### EXAMPLE 15 p53 VARIANT (SEQ ID NO.3) PROTEIN OF THE PRESENT DISCLOSURE HAS IMPROVED ABILITY TO KILL HUMAN NEUROBLASTOMA CELL LINE SH-SY5Y

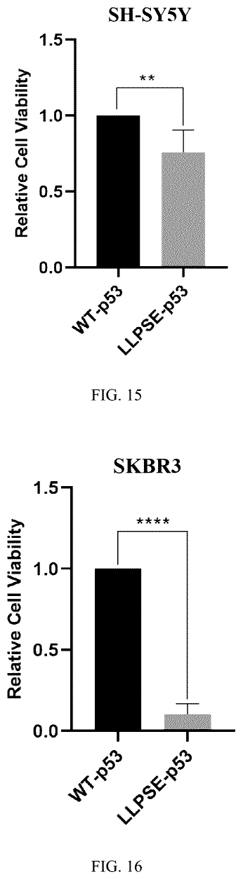

Verification was further performed at a protein level on SH-SY5Y cells, and it was found that LLPSE-p53 recombinant protein also had significant killing effect on SH-SY5Y tumor cells, and the killing effect was stronger than that of WT-p53 (FIG. 15).

Implementation was made as follows:

Specific implementation steps can refer to Example 14.

Results show that for SH-SY5Y tumor cells, LLPSE-p53 recombinant protein has a stronger pro-apoptotic effect on SH-SY5Y tumor cell compared with WT-p53 recombinant protein.

### EXAMPLE 16 LLPSE-p53 (SEQ ID NO.3) RECOMBINANT PROTEIN ENHANCES KILLING EFFECT OF p53 ON HUMAN BREAST CANCER CELL LINE SKBR3

Verification was further performed at a protein level on SKBR3 cells, and it was found that LLPSE-p53 recombinant protein also improved killing effect on SKBR3 tumor cells over WT-p53 recombinant protein (FIG. 16).

Implementation was made as follows:

Specific implementation steps can refer to Example 14.

The results show that the LLPSE-p53 recombinant protein has a stronger pro-apoptotic effect on SKBR3 tumor cells compared with WT-p53.

### EXAMPLE 17 LLPSE-p53 (SEQ ID NO.3) RECOMBINANT PROTEIN ENHANCES KILLING EFFECT OF p53 ON HUMAN OSTEOSARCOMA CELL LINE U-2OS

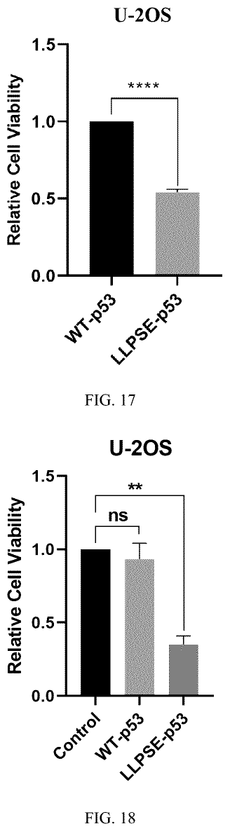

Verification was further performed on U-2OS cells at a protein level, and it was found that LLPSE-p53 recombinant protein also had improved killing effect on U-2OS tumor cells over WT-p53 recombinant protein (FIG. 17).

Implementation was made as follows:
1. 5000 cells/well were seeded in a 96-well cell culture plate, 100 μl of complete medium was added (with no P/S), the cells were then cultured in an incubator for 12-24 h.
2. 50 μl of protein delivery system was prepared according to a protein final concentration of 6.5-10 μM as follows:
6.5-10 μM of protein+5×transduction buffer+complete medium (no P/S).
3. The above system was sterilized with a 0.22 μm filter membrane.
4. The complete medium in the well plate was sucked and discarded, and the above protein delivery system was added, the plate was then placed in an incubator for culture for 24 h.
5. The protein delivery system was sucked and discarded, and complete medium was added, which was then cultured for 24 h.
6. Cell viability was measured by a CCK8 kit and with a M5 microplate reader.
8. Graphpad5 software was used for data processing and analysis.

The results show that the LLPSE-p53 recombinant protein has a stronger tumor killing effect compared with WT-p53 in U-2OS tumor cells.

### EXAMPLE 18 LLPSE-p53 (SEQ ID NO.3) RECOMBINANT PROTEIN HAS BETTER APPLICATION PROSPECTS THAN WILD-TYPE p53 RECOMBINANT PROTEIN IN TUMOR TREATMENT

To investigate whether LLPSE-p53 offers greater potential for clinical tumor treatment than WT-p53, recombinant proteins of both WT-p53 and LLPSE-p53 were separately introduced into U-2OS tumor cells, alongside a control group that did not receive any protein. Results show that the WT-p53 recombinant protein does not inhibit proliferation of U-2OS cells, while the LLPSE-p53 recombinant protein significantly inhibits the proliferation of U-2OS cells (FIG. 18).

Implementation was made as follows:

Specific implementation steps can refer to Example 17.

The data show that LLPSE-p53 has better application prospect in treatment of osteosarcoma than WT-p53.

### EXAMPLE 19 LLPSE-p53 (SEQ ID NO.3) RECOMBINANT PROTEIN ENHANCES KILLING EFFECT OF p53 ON HUMAN BRAIN TUMOR CELL LINE SF126

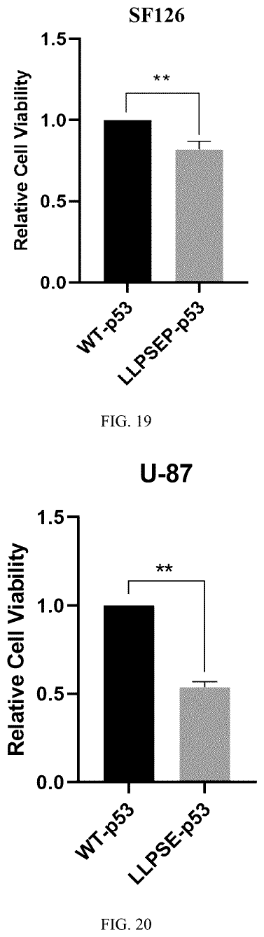

Verification was further performed on SF126 cells at a protein level, and it was found that LLPSE-p53 recombinant protein also improved killing efficiency on SF126 tumor cells over WT-p53 recombinant protein (FIG. 19).

Implementation was made as follows:

Specific operation steps can refer to Example 17.

The data show that for SF126 tumor cells, LLPSE-p53 recombinant protein has improved ability to inhibit proliferation of SF126 tumor cells compared with WT-p53 recombinant protein.

### EXAMPLE 20 LLPSE-p53 (SEQ ID NO.3) RECOMBINANT PROTEIN ENHANCES KILLING EFFECT OF p53 ON HUMAN MALIGNANT GLIOMA CELL LINE U-87

Inhibitory effect of LLPSE-p53 recombinant protein on proliferation of tumor cells was measured in a human malignant glioblastoma U-87 cell line, and it was found that LLPSE-p53 has more significant killing efficiency on U-87 cells than WT-p53 (FIG. 20).

Implementation was made as follows:

Specific implementation steps can refer to Example 17.

The data show that LLPSE-p53 recombinant protein has stronger killing effect on U-87 cells than WT-p53 recombinant protein.

### EXAMPLE 21 LLPSE-p53 (SEQ ID NO.3) EXERTS ITS TUMOR KILLING FUNCTION BY REGULATING FGFR3

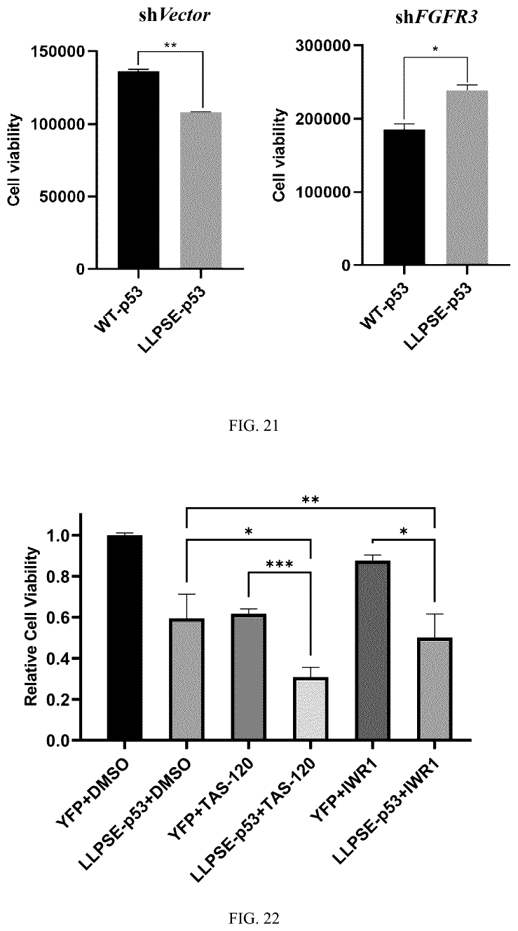

In order to further explore mechanism through which LLPSE-p53 suppresses cancer cell growth, an RNA-seq screening was conducted, and it was found that over-expression of LLPSE-p53 leads to a reduction in the mRNA level of FGFR3 among others, in comparison to p53. After knocking down of FGFR3, it was found that LLPSE-p53's tumor suppression capability was diminished, indicating that LLPSE-p53 inhibited proliferation of the tumor cells through FGFR3 (FIG. 21), and suggesting that treatment effect of LLPSE-p53 in tumors with a high expression level of FGFR3 gene is more remarkable.

Implementation was made as follows:
1. 5×10⁵-6×10⁵ cells were seeded into a 12-well cell culture plate, and 1 ml of complete medium was added for culture for 12 to 24 h.
2. 1 μg of shVector and 1 μg of shFGFR3 plasmids were transfected into cells respectively, the cells were then placed in an incubator for culture for 48 h.
3. Above two groups of cells were detached and counted, and 5000 cells per well were seeded into a 96-well plate, the plate was then cultured for 24 h.
4. 100-200 ng of plasmids were transfected into each well, the plate was then cultured in an incubator for 72 h.
5. Cell viability was measured by an ATP method and with a M5 microplate reader.
6. Graphpad5 software was used for data processing and analysis.

Results show that ability of LLPSE-p53 to inhibit cell activity is stronger than that of WT-p53 without knocking down of FGFR3 gene. When FGFR3 is knocked down, the ability of LLPSE-p53 to inhibit cell activity is lost, indicating that LLPSE-p53 exerts its tumor killing function via FGFR3.

### EXAMPLE 22 THE EFFICACY OF LLPSE-p53 (SEQ ID NO.3) IN KILLING H1299 CELLS IS ENHANCED WHEN COMBINED WITH TAS-120 OR IWR-1

To investigate whether tumor-killing capability of LLPSE-p53 can improve by combined with small-molecular inhibitors at gene level, LLPSE-p53 and YFP plasmids were transfected respectively into H1299 cells and then incubated with FGFRs inhibitor TAS-120 or Wnt signaling pathway inhibitor IWR-1. The results show that LLPSE-p53, in combination with TAS-120 and IWR-1, substantially boosts its ability to eliminate H1299 cells. The data suggest that at the gene level, the combination of LLPSE-p53 with TAS-120 or IWR-1 tumor inhibitors can enhance the tumor suppression effect of LLPSE-p53 (FIG. 22), highlighting its potential for clinical cancer therapy. Implementation was made as follows:
1. 5000 cells/well were seeded into a 96-well cell culture plate, 100 μl of complete medium was added to each well, the cells were then placed in an incubator for continuous culture for 12-24 h.
2. 100 ng to 200 ng of plasmid was transfected into cells, with 3 wells in each group, the plate was then placed in an incubator for 24 h.
3. 5-10 μM of TAS-120 or IWR-1 was added to the transfected cells, and the same amount of DMSO (dimethyl sulfoxide) was added as a control, then the cells were incubated in CO₂ incubator at 37° C. for 48 h.
3. Cell proliferation was measured by a CCK8 method.
4. Graphpad5 software was used for data processing and one-way ANOVA method was used for statistical analysis.

Results show that combining LLPSE-P53 with TAS120 or IWR-1 can significantly improve the effectiveness of LLPSE-p53 in killing tumor cells.

### EXAMPLE 23 THE EFFICACY OF TAS-120 OR IWR-1 IN KILLING H1299 CELLS IS ENHANCED WHEN COMBINED WITH LLPSE-p53 (SEQ ID NO.3) PROTEIN

To further explore feasibility of LLPSE-p53 in clinical application, LLPSE-p53 recombinant protein was used in combination with a FGFR inhibitor TAS-120 or a Wnt signaling pathway inhibitor IWR-1 to treat U-2OS cells. Results show that LLPSE-p53 significantly promotes TAS-120 or IWR-1 to kill U-2OS cells, when compared with a case in which TAS-120 or IWR-1 are combined with the buffer (blank control), indicating that LLPSE-p53 can enhance the tumor killing ability of TAS-120 or IWR-1 (FIG. 23).

Implementation was made as follows:
1. 5000 cells/well were seeded into a 96-well cell culture plate, 100 μl of complete medium was added (with no P/S), the plate was then placed in an incubator for culture for 12 to 24 h.
2. 50 μl of protein delivery system was prepared according to a protein final concentration of 0.5-2 μM as follows:
0.5-2 μM of protein+5×transduction buffer+complete medium (no P/S).
3. The above system was sterilized with a 0.22 μm filter membrane.
4. The complete medium in the well plate was sucked and discarded, and the above protein delivery system was added, the plate was then placed in an incubator for culture for 24 h.
5. The protein delivery system was sucked and discarded, complete medium containing TAS-120 and IWR-1 with a final concentration of 5-20 μM was added respectively, with a same volume of DMSO was added as a control, the plate was then cultured for 24 h.
6. Cell viability was measured by a CCK8 method and with a M5 microplate reader.
7. Graphpad5 software was used for data processing and one-way ANOVA method was used for statistical analysis.

### EXAMPLE 24 A PHASE SEPARATION ENHANCING SEQUENCE IN LLPSE-p53 HAS AN ADVANTAGE OF PROMOTING PHASE SEPARATION

In order to prove that unique phase separation enhancing sequence encompassed by the formula in this disclosure can have positive effect, pCMV6-p53, pCMV6-LLPSE-p53, pCMV6-LLPSE-p53 (ΔH), pCMV6-LLPSE-p53 (ΔR), pCMV6-LLPSE-p53R11, pCMV6-LLPSE-p53 (ΔHR2H3R2), pCMV6-LLPSE-p53 (ΔHR11), and pCMV6-LLPSE-p53 (ΔHR9H8R9) eukaryotic expression vectors were constructed, and were transfected into H1299 cell line, and phase separation ability of the above variants were compared. Results show that the phase separation enhancing sequence in the formula listed in the disclosure has good ability to promote the phase separation of p53 (FIG. 24).

Implementation was made as follows:

Specific implementation steps can refer to Example 3.

Results show that the phase separation enhancing sequence in LLPSE-p53 can promote the phase separation of p53 well.

### EXAMPLE 25 A PHASE SEPARATION ENHANCING SEQUENCE IN LLPSE-p53 HAS AN ADVANTAGE OF IMPROVING TRANSCRIPTIONAL ACTIVATION ACTIVITY OF p53

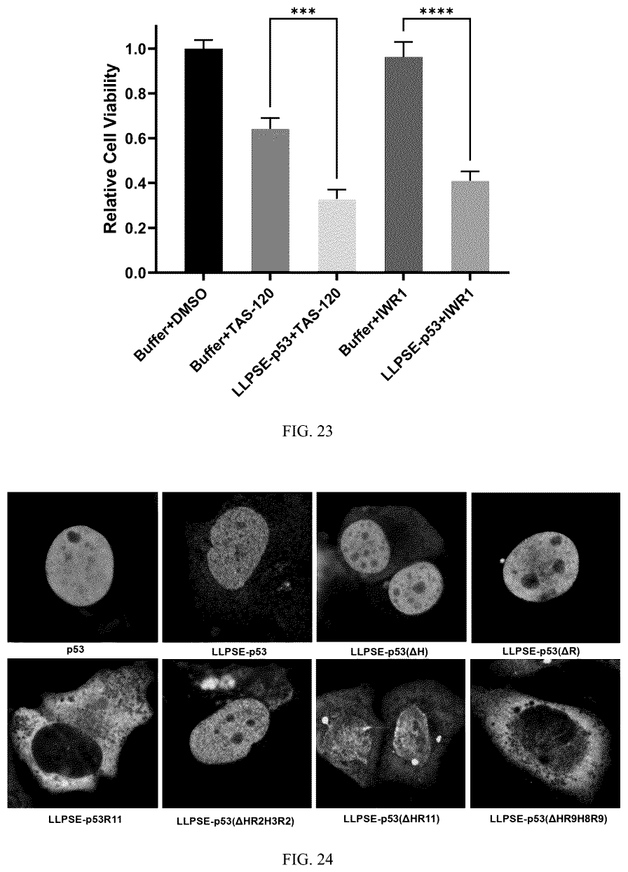

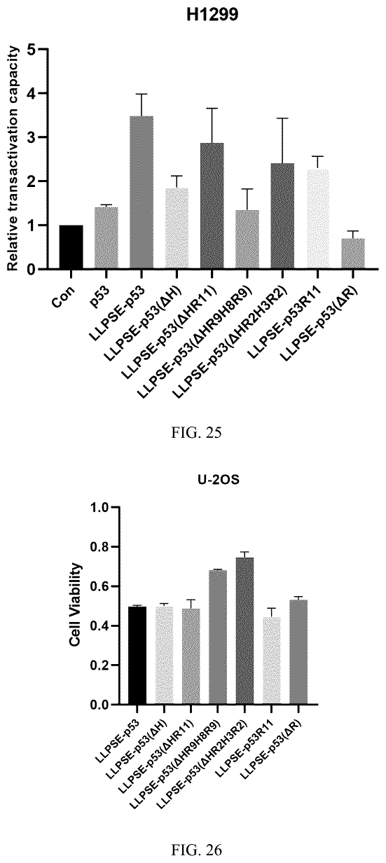

The influence of amino acid composition on p53 transcription activation activity was explored for different sequences, some of which are included in the formula listed in this disclosure. pCMV6-p53, pCMV6-LLPSE-p53, pCMV6-LLPSE-p53(ΔH), pCMV6-LLPSE-p53(ΔR), pCMV6-LLPSE-p53R11, pCMV6-LLPSE-p53(ΔHR2H3R2), pCMV6-LLPSE-p53(ΔHR11), pCMV6-LLPSE-p53(ΔHR9H8R9) eukaryotic expression vectors were constructed, and were transfected into H1299 cell line, and transcriptional activation of CDKN1A by p53 variants described above were measured. Results show that the phase separation enhancing sequence in LLPSE-p53 is optimal in enhancing transcriptional activation ability of p53 (FIG. 25).

Implementation was made as follows:

Specific implementation steps can refer to Example 8.

Results show that when H and R (ΔH and ΔR) are deleted, ability of transcriptional activation of target gene CDKN1A is weakened. Both histidine (H) and arginine (R) in an exogenous sequence added are necessary to promote the liquid-liquid phase separation and for stronger transactivation activity of p53, and the phase separation enhancing sequence in LLPSE-p53 has optimal effect of improving the transcriptional activation ability of p53.

### EXAMPLE 26 THE PHASE SEPARATION ENHANCING SEQUENCES IN LLPSE-p53 SIGNIFICANTLY BOOST THE p53's CAPABILITY TO SUPPRESS THE PROLIFERATION OF HUMAN OSTEOSARCOMA CELL LINE, U-2OS

Different sequences enriched in positive charge or histidine were used to check whether liquid-liquid phase separation enhancing sequence in the formula listed in the present disclosure are more effective in inhibiting cancer cell proliferation. Effects of p53 variants with these different sequences on inhibition of proliferation of tumor cells by p53 were compared in the U-2OS cell line. pCMV6-LLPSE-p53, pCMV6-LLPSE-p53(ΔH), pCMV6-LLPSE-p53(ΔR), pCMV6-LLPSE-p53R11, pCMV6-LLPSE-p53(ΔHR2H3R2), pCMV6-LLPSE-p53(ΔHR11), pCMV6-LLPSE-p53(ΔHR9H8R9) eukaryotic expression vectors were constructed, and were transfected into U-2OS cells, and cell proliferation was measured. Results show that p53 variants with liquid-liquid phase separation enhancing sequence in the formula listed in the present disclosure have better ability to inhibit the proliferation of tumor cells (FIG. 26).

Implementation was made as follows:

Specific implementation steps can refer to Example 12.

Results show that LLPSE-p53 has strong ability to inhibit the proliferation of U-2OS cells.

EXAMPLE 27 LIQUID-LIQUID PHASE SEPARATION ENHANCING SEQUENCES CORRESPONDING TO THE SEQUENCE IN THE FORMULA LISTED IN THE PRESENT DISCLOSURE HAVE PREDOMINANTLY UNIFORM SPATIAL STRUCTURAL CHARACTERISTICS

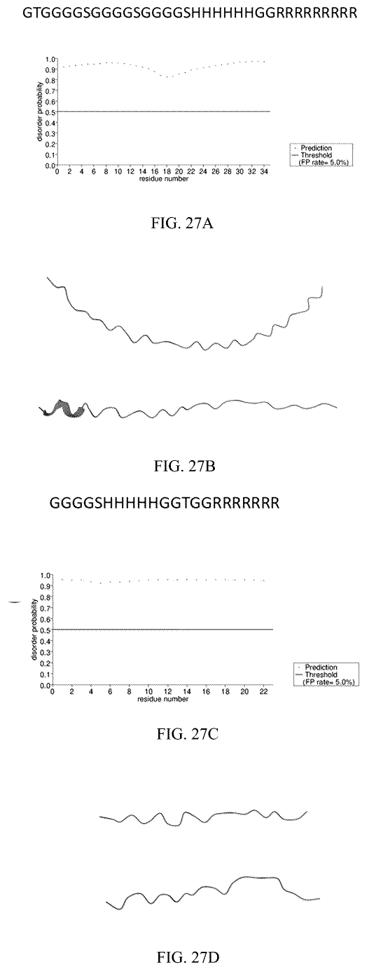

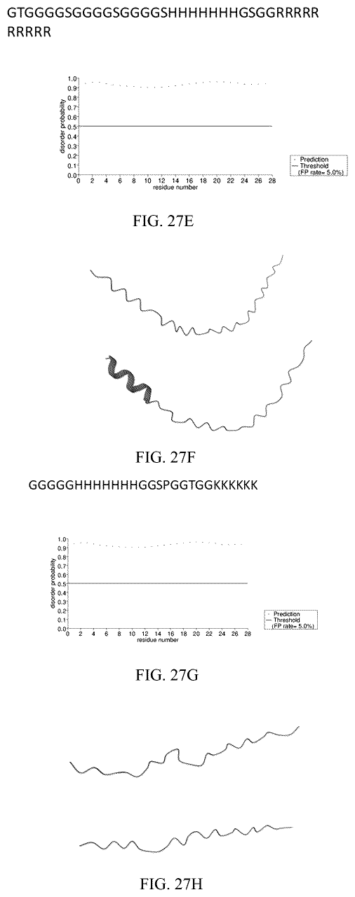

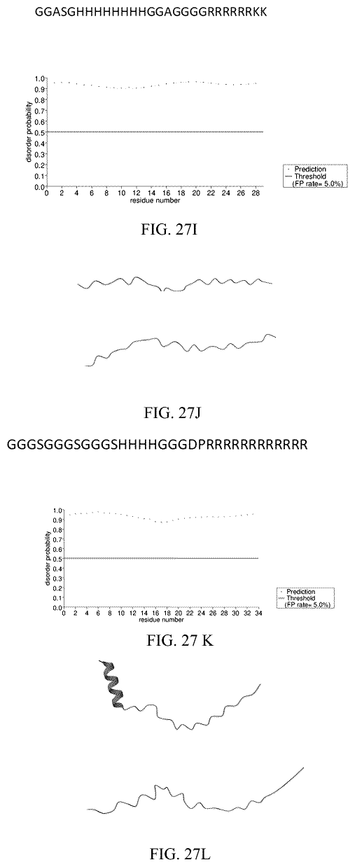

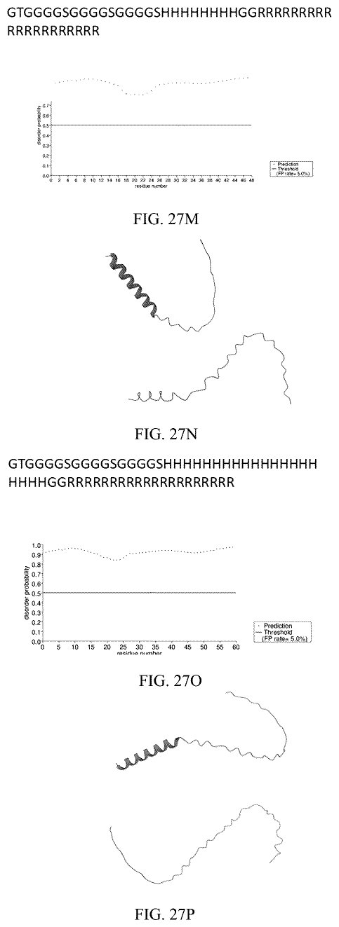

It was further explored whether the liquid-liquid phase separation enhancing sequences in the formula listed in the disclosure have a uniform spatial structure, the spatial structures were predicted based on the amino acid sequences. Results show that the phase separation enhancing sequence contained in the disclosure's formula exhibit a predominantly uniform structure (FIGS. 27A-27P).

The above-described embodiments only express several implementations of the present invention, and their descriptions are more specific and detailed, but they cannot be constructed as limiting the scope of the present disclosure. It should be noted that, several modifications and improvements can be made by those of ordinary skills in the art without departing from the concept of the present invention, which belong to the protection scope of the present invention. Therefore, the protection scope of this disclosure shall be subjected to the appended claims

What is claimed is:

1. A p53 variant with enhanced liquid-liquid phase separation ability, wherein the p53 variant comprises an amino acid sequence rich in positive charges and histidine at one terminus of a p53 sequence, which has stronger liquid-liquid phase separation ability and stronger transactivation activity in cells and in vitro than the p53 sequence; and
a general formula of the amino acid sequence rich in positive charges and histidine is Hx(G/S/P/A/T)y(R/K)z, where H represents histidine; (G/S/P/A/T) represents a sequence composed of one or more of glycine, serine, alanine, proline, and threonine; and (R/K) represents arginine or lysine, where x, y and z represent numbers of respective amino acids; x and z range from 0 to 20; y ranges from 0 to 100, and x+z>5.

2. The p53 variant according to claim 1, wherein a linker sequence is further connected between the amino acid sequence rich in positive charges and histidine, and the terminus of the p53 sequence.

3. The p53 variant according to claim 1, a DNA sequence of the p53 is shown in SEQ ID NO.1 or SEQ ID NO.2.

4. The p53 variants according to claim 1, wherein the p53 variants have transactivation activity.

5. The p53 variant according to claim 1, wherein a DNA sequence of the p53 variant is shown in SEQ ID NO.3, SEQ ID NO.4, SEQ ID NO.5, SEQ ID NO.6, SEQ ID NO.7 or SEQ ID NO.8.

6. The p53 variant according to claim 1, wherein the p53 variant activates transcription of CDKN1A and improves mRNA levels of one or more of downstream target genes CDKN1A, MDM2, PUMA, NOXA and RRM2B.

7. An application of the p53 variant according to claim 1 in preparing a drug for treating tumors, wherein an applicable tumor type involves non-small cell lung cancer, breast cancer, neuroblastoma, osteosarcoma, and human brain tumor;
and the drug comprises a p53 variant in either nucleic acid or protein form.

8. The application according to claim 7, wherein a recombinant protein expression system of the p53 variant comprises Escherichia coli. expression host, eukaryotic cell and yeast protein expression systems; prokaryotic expression vectors comprise pET24a and pET28a (+), and eukaryotic expression vectors comprises: 1) eukaryotic cell expression vectors comprising pEGFP, pEYFP, pmcherry, pRFP, pECFP, pLenti, pLX, pCMV6, pCMV3, pcDNA3 and pcDNA6B; 2) insect cell expression vectors comprising pAc5.1-EGFP; and 3) yeast expression vectors comprising pPIC3 and pPIC9.

9. The application according to claim 7, wherein the protein form of the p53 variant comprises either a full-length form or a mutant form that retains functions.

10. The application according to claim 7, wherein a target for tumor-cell apoptosis mediated by the p53 variant comprises FGFR3, and the p53 variant is configured for treating tumors with high FGFR3 expression.

11. The application according to claim 7, wherein a drug for treating tumors that combines the p53 variant, with a FGFR inhibitor TAS-120 or a Wnt signal pathway inhibitor IWR-1, or a prepared composite drug, for improving its ability to kill the tumor cells.

12. The application according to claim 7, wherein the drug is in a form of injection, tablet, capsule, oral liquid dosage, granule, or ointment.


SEQUENCE LISTING (8 sequences)

SEQ ID NO: 1 - DNA, 1179 bp, Homo sapiens
  source 1..1179: mol_type = genomic DNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagctgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgctgagtatttggatgacagaaacacttttcgacatagtgtggtggtgccctatgagccgcctgaggttggctctgactgtaccaccatccactacaactacatgtgttacagttcctgcatgggcggcatgaaccggaggcccatcctcaccatcatcacactggaagactccagtggtaatctactgggacgggacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagac

SEQ ID NO: 2 - DNA, 1179 bp, Homo sapiens
  source 1..1179: mol_type = genomic DNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagac

SEQ ID NO: 3 - DNA, 1281 bp, synthetic construct
  source 1..1281: mol_type = other DNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagctgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgctgagtatttggatgacagaaacacttttcgacatagtgtggtggtgccctatgagccgcctgaggttggctctgactgtaccaccatccactacaactacatgtgttacagttcctgcatgggcggcatgaaccggaggcccatcctcaccatcatcacactggaagactccagtggtaatctactgggacgggacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagacggtaccggtggaggaggttctggaggcggtggaagtggtggcggaggtagccaccaccaccaccaccacggtggtcgtcgtagacgtcgtcgtcgacgtcgt

SEQ ID NO: 4 - DNA, 1245 bp, synthetic construct
  source 1..1245: mol_type = other DNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagctgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgctgagtatttggatgacagaaacacttttcgacatagtgtggtggtgccctatgagccgcctgaggttggctctgactgtaccaccatccactacaactacatgtgttacagttcctgcatgggcggcatgaaccggaggcccatcctcaccatcatcacactggaagactccagtggtaatctactgggacgggacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagacggtggcggaggtagccaccaccaccaccacggcggtaccggtggtcgtcgtagacgtcgtcgtcga

SEQ ID NO: 5 - DNA, 1293 bp, synthetic construct
  source 1..1293: mol_type = other DNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagacggtaccggtggaggaggttctggaggcggtggaagtggtggcggaggtagccaccaccaccaccaccaccacggtagtggtggccgtcgtagacgtcgtcgtcgacgtcgtcgt

SEQ ID NO: 6 - DNA, 1260 bp, synthetic construct
  source 1..1260: mol_type = other DNA
atggaggagccgcagtcagatcctagcgtcgagccccctctgagtcaggaaacattttcagacctatggaaactacttcctgaaaacaacgttctgtcccccttgccgtcccaagcgatggatgatttgatgctgtccccggaagatattgcacaatggttcactgaagacccaggtccacatgaagctcccagaatgtcagaggctgctccccccatggcccccgcaccaggagctcctacactggcggcgcctgcaccagccccctcctggcccctgtcatcctctgtcccttcccagaaaacctaccagggcagctacggtttccgtctgggcttcttgcattctggaacggccaagtctgtgacttgcacgtactcccctgccctcaacaagatgttttgccagctggccaagacctgccctgtgcagctgtgggttgattccacaccccctcccggcacccgtgtccgcgccatggccatctacaagcagtcacagcacatgacggaggttgtgaggcgctgcccccaccatgagcgctgctcagatagcgatggtctggcccctcctcagcatcttatccgagtggaaggcaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgcgtttgtgcctgtcctgggagagaccggcgcacagaggaagagaatttccacaagaaaggggagcctcaccacgagctgccccctgggagcactaagcgagcactgcccaacaacaccagctcctctccccagccaaagaagaaaccactggatggagaatatttcacccttcagatccgtgggcgtgagcgcttcgagatgttccgagagctgaatgaggctttggaactcaaggatgcccaggctgggaaggagccaggggggagcagggctcactccagccacctgaagtccaagaagggtcagtctacctcccgccataaaaaacttatgttcaagacagaagggcctgactcagacggcggtggcggcggccaccaccaccaccaccaccacggcggcagccccggcggcaccggcggcaagaagaagaagaagaag

SEQ ID NO: 7 - DNA, 1257 bp, synthetic construct
  source 1..1257: mol_type = other DNA
atggaggattcacagtcggatatgagcatcgagctccctctgagtcaggagacattttcatgcttatggaaacttcttcctccagatgatattctgcccaccacagcgacagggtcacctaattccatggaagatctgttcctgccccaggatgttgcagagttgttagaaggcccagaggaagccctccaagtgtcagctcctgcagcacaggaacctggaactgaggcccctgcacccgtggcccctgcttcagctacaccgtggcctctgtcatcttccgtcccttctcaaaaaacttaccaaggcaactatggcttccacctgggcttcctgcagtcagggacagccaagtctgttatgtgcacgtactcaatttccctcaataagctgttctgccagctggcgaagacatgccctgtgcagttgtgggtcacctccacacctccacctggtacccgtgtccgtgccatggccatctacaagaagtcacaacacatgactgaggtcgtgagacgctgcccccaccatgagcgttgctctgatggtgacggcctggctcctccccaacatcttatccgggtggaaggaaatccgtatgctgagtatctggacgacaggcagacttttcggcacagcgtggtggtaccgtatgagccacctgaggtcggctccgactataccactatccactacaagtacatgtgcaacagctcctgcatggggggcatgaaccgccggcccatccttaccatcatcacgctggaagactccagtgggaatcttctgggacgggacagctttgaggttcgtgtttgtgcctgtcctgggagagaccgtcggacagaggaagaaaatttccgcaaaaaagaagagcattgcccggagctgcccccagggagtgcaaagagagcactgcccaccagcacaagctcctctccccagcaaaagaaaaaaccactcgatggagaatatttcacccttaagatccgtgggcgtgagcgcttcgagatgttccgagagctgaatgaggccttggaattaaaggatgcccgtgctgccgaggagtcaggagacagcagggctcactccagctacccgaagaccaagaagggccagtctacgtcccgccataaaaaaccaatgatcaagaaagtggggcctgactcagacggaggcgcaagtggtcaccaccaccaccaccaccaccacggcggcgccggcggcggcggcaggaggaggaggaggaggaagaag

SEQ ID NO: 8 - DNA, 1380 bp, synthetic construct
  source 1..1380: mol_type = other DNA
atgtaccctcccctagcaggtcctctgcctagctctctgactagggttggctatatctcacacccagtcctcatttttcccctcatagcagccttcttagctgctgccatggaagagccacagtcggatctcagcatcgagcctccactgagtcaggagacattttcagacttatggaaactacttcctgaaaacaacgttctgtccagctcactgtcctctcccatggatgatctgctgctgtccccagaagatgttgtaaactggctgggaggaaacccagatgaagatgtccaagtgtcagcagctcctgtaccagagcccccaacaccagtggcccctgccccggcagctcccgcaccagccacttcctggcctctgtcatcctccgtcccttcccataagacctaccaaggcaactatggtttccatctgggcttccttcagtctgggacggccaaatctgtcacatgcacgtactcccctgttctcaacaagttattctgccaactggcaaagacctgccctgtgcaagtgtgggtcgaatcaccacccccacctggcacccgagtccgtgccatggccatctacaagaagtcacagcacatgacagaagttgtgaggcgctgcccccaccatgagcgctgctccgatagtgatggcctggcccctcctcagcatcttatccgggtggaaggaaatctgcgtgcagaatatttggatgacagaaccacttttcgccatagcgtggtggtaccctatgatctgcctgaggttggctctgactgtaccaccatccactacaactatatgtgcaacagttcttgcatggggggcatgaaccgtaggcccatcctcaccattatcacactggaagactccagtgggaacctgctggggcggaacagctttgaggtgcgtgtttgtgcctgtcctgggagagaccggcgcacagaggaagaaaatttccacaagaaaggggggtcatgcccagagccaacaccaggaagcattaagcgagcactgcccactggcaccaactcttctcctcagccaaagaagaaaccactggatggggaatatttcacccttaagatccgtgggcgtgaacgctttgagatgttccgagagctaaatgaggccttggaactcaaggatgcccaaactgagaaggagccaggggagagcaggcctcactcaagctacctgaagtctaagaaggggcagtctacctcctgtcataaaaaactaatgttcaagaaagaaggacctgattcagacggagggggttccggtggcggttccggtggcggttctcaccaccaccacggtggcggggatccacgtcgtagacgtcgtcgtcgacgtcgtagacgtcgt